(19) United States
(12) Patent Application Publication
(10) Pub. No.: US 2017/0172921 A1
(43) Pub. Date: Jun. 22, 2017

(54) POLYPHENOLIC COMPOUNDS ENCAPSUALATED IN LONG CIRCULATING LIPOSOMES AND USE THEREOF

(71) Applicants: Mahmoud Reza Jaafari, Mashhad (IR); Fatemeh Gheybi, Mashhad (IR); Seyed Mahdi Rezayat Sorkhabadi, Tehran (IR)

(72) Inventors: Mahmoud Reza Jaafari, Mashhad (IR); Fatemeh Gheybi, Mashhad (IR); Seyed Mahdi Rezayat Sorkhabadi, Tehran (IR)

(21) Appl. No.: 15/378,091

(22) Filed: Dec. 14, 2016

(51) Int. Cl. A61K 9/127

(52) U.S. Cl. CPC A61K 9/127; A61K 9/1277

(57) ABSTRACT

A method for preparing a liposome composition for delivering polyphenolic compounds to the target organs may include preparing a complex that contains at least one polyphenolic compound. Then, encapsulating the polyphenolic compound into a liposome composition by admixing the prepared complex with the liposome ingredients.

### TECHNICAL FIELD

The present invention generally relates to delivering a polyphenolic compound, specifically to enhancing the cellular uptake of polyphenolic compounds, and more specifically preparing a liposome composition for stably incorporating high content of polyphenolic compounds therein.

### BACKGROUND

Today, there is increasing interest in finding naturally occurring chemo-preventive agents that may have different mechanisms of action with non-overlapping toxicity. Polyphenols, and their derivatives exist in nature and offer numerous health benefits. Besides being potent antioxidants, some polyphenols have other biological activities that may prevent certain diseases such as inhibition of tumor growth, improvement of vascular function, and modulation of the immune system.

In the medical field, some polyphenolic derivatives, such as flavonoids, may interact with cells and influence specific signaling pathways, which may result in hindering angiogenesis or inhibit tumor growth.

However, since the polyphenolic compounds generally have low water solubility and lack easily ionizable groups, they have reduced bioavailability and consequently, their application is limited. Therefore, there is a need in the art for a method to improve the bioavailability of polyphenolic compounds through increasing the water solubility of polyphenolic compound without any significant change to the common fabrication processes of medicine.

### SUMMARY

The following brief summary is not intended to include all features and aspects of the present disclosure, nor does it imply that any exemplary embodiments must include all features and aspects discussed in this summary.

In on general aspect, the present disclosure describes a method for preparing a liposome composition containing at least one polyphenolic compound. An exemplary implementation of the method may include the steps of: first, forming a complex by reacting at least one polyphenolic compound with a first phospholipid and a solvent or a mixture of solvents, second, admixing the prepared complex with at least one second phospholipid, cholesterol, one or more antioxidant and a PEG-Phospholipid; third, preparing multi-lamellar vesicles using the admixture to form, dissolving the above admixture in to a solvent and removing organic solvents to prepare lipid film; hydrating the lipid film to form multi-lamellar vesicles and finally, downsizing the formed multi-lamellar vesicles to reach the nano-sized liposome composition.

The above general aspects may include one or more of the following features. The prepared liposome may be intravenously administrated. In an implementation, the solvent may be selected from acetone, dioxin, n-hexane, any other aprotic solvents, or a mixture thereof. In another implementation, the complex may be isolated by, for example lyophilization, spray-drying, and precipitation with non-solvents (e.g., aliphatic hydrocarbons).

In an exemplary implementation, the first and the second phospholipids may be selected from phosphatidylcholine (PC), phosphatidylethanolamine (PE), or phosphatidylserine (PS). The term “first phospholipid” is one or more than one phospholipid that is approved for use in forming the complex. In an exemplary implementation, the first phospholipid may be soybean phosphatidycholine (SPC).

The second phospholipid may be selected from egg phosphatidylcholine (EPC), SPC, hydrogenated SPC (HSPC), Dipalmitoylphosphatidylcholine (DPPC), or dimyristoylphosphatidylcholine (DMPC).

The term “second phospholipid” used herein in this application, refer to the phospholipid molecule that may be used for preparing the liposome. In an example implementation, one or more than one abovementioned phospholipids may be used for preparing the liposome according to present disclosure.

According to some implementations, the PEG-phospholipid may be 1,2-distearoyl-sn-glycero-3-phosphoethanolamine-N-[amino (polyethylene glycol)-X] (mPEGX-DSPE), in which the X may be in a range of about 500 to about 5000.

In some implementations, the molar ratio of the first phospholipid to the at least one poly phenolic compound may be for example in a range of 0.5:1 to 3:1 or 1:2 to 1:3. The molar ratio in some implementation may be selected to the number of OH groups on polyphenolic compounds.

In other implementations, the molar ratio of the liposome ingredients including prepared complex:second phospholipid:cholesterol:PEG-phospholipid:and antioxidant may be in a range of 2:20:5:1:0.02 to 3:25:6:2:0.08.

In some example implementations, the polyphenolic compound may be selected from anthocyanidins, catechins, flavonoids (flavanones, flavones, flavonols, isoflavones, etc.), hydroxybenzoic acids, hydroxycinnamic acids, lignans, stilbenes, tannins (proanthocyanidines), monophenols, capsaicinoids, curcumin, or derivatives thereof,

According to another exemplary implementation, preparing the lipid film may take place via dissolving the admixture of the formed complex, second phospholipid, cholesterol, antioxidants, and the PEG-phospholipid into an organic compound that may be for example chloroform, and removing the organic solvents.

In an exemplary operation, removing the organic solvents may be carried out via rotary evaporation and freeze-dried operation. According to further exemplary implementations, multi-lamellar vesicles (MLVs) may be prepared by hydrating and dispersing the prepared lipid film with a hydrating liquid. In some example implementations, the hydrating liquid used for preparing the MLVs may be HEPES. Further operations for preparing MLVs may include one or all of following exemplary operations including sonicating, extruding, filtering, and freeze drying.

### BRIEF DESCRIPTION OF THE DRAWINGS

This application will be understood more clearly from the following description and the accompanying figures. These figures are given purely by way of an indication and in no way restrict the scope of the application. Of these figures:

FIG. 1 illustrates an example of a method for preparing a liposome composition, according to one or more aspects of this disclosure.

FIG. 2A illustrates the results of tumor size monitoring experiments in female BALB/c mice bearing 4T1 breast tumor treated with silybin liposome formulation, described in detail in connection with Example 6.

FIG. 2B illustrates the results of tumor size monitoring experiments in female BALB/c mice bearing 4T1 breast tumor treated with silymarin liposome formulation, described in detail in connection with Example 6.

FIG. 2C illustrates the results of tumor size monitoring experiments in female BALB/c mice bearing TUBO breast tumor treated with silybin liposome formulation alone or in combination with Doxil (doxorubicin liposomal formulation) according to one or more aspects of this disclosure.

FIG. 3A illustrates the result of in vivo survival test in female BALB/c mice bearing 4T1 breast tumor treated by silybin liposomal formulations, described in detail in connection with Example 6.

FIG. 3B illustrates the result of in vivo survival test in female BALB/c mice bearing 4T1 breast tumor treated by silymarin liposomal formulations, described in detail in connection with Example 6.

FIG. 3C Illustrates the result of in vivo survival test in female BALB/c mice bearing TUBO breast tumor treated by silybin liposomal formulation alone or in combination with Doxil (doxorubicin liposomal formulation) according to one or more aspects of this disclosure.

### DETAILED DESCRIPTION

The following detailed description is presented to enable persons of ordinary skill in the art to make and use the teachings of the instant application. For purposes of explanation, specific examples are described to assist ones of skill in the art in readily understanding the concepts disclosed in the present application. However, it will be apparent to one skilled in the art that these specific details are not required to practice the teachings of the instant application. Descriptions of specific applications are provided only as representative examples. Various modifications to the described implementations will be readily apparent to one skilled in the art upon reading this disclosure, and the general principles defined herein may be applied to other implementations and applications without departing from the scope of the present application. The present application is not intended to be limited to the implementations shown, but is to be accorded the widest possible scope consistent with the principles and features disclosed herein.

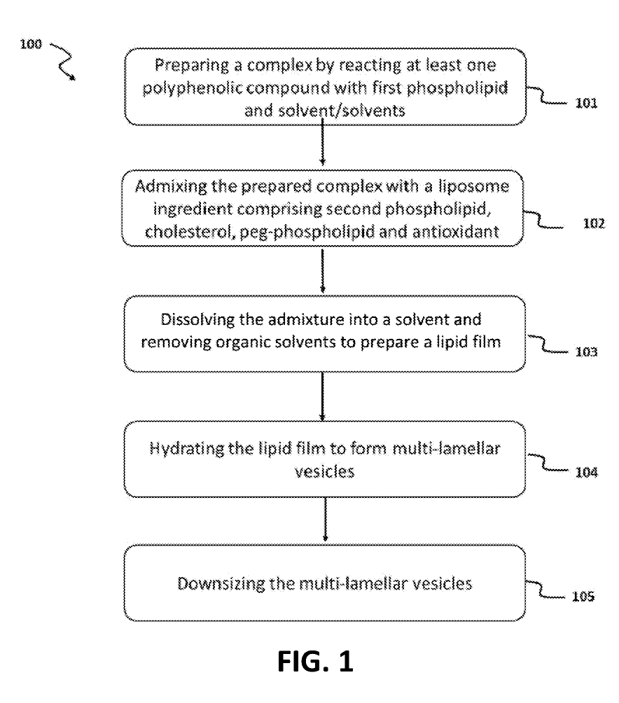

Exemplary implementations consistent with the present disclosure include a method for preparing a liposome composition for delivering at least one polyphenolic compound. In exemplary embodiments, the liposome composition is delivered intravenously. Referring now to FIG. 1, an exemplary method 100 for preparing liposome-encapsulated polyphenolic compound may include the steps of: forming a complex by reacting the at least one type of polyphenolic compound with a first phospholipid and an organic solvent (step 101); Admixing the prepared complex with a liposome ingredient that includes a second phospholipid, cholesterol, a PEG-phospholipid and an antioxidant in order to obtain an admixture (step 102); dissolving the admixture into a solvent and removing the organic solvent to prepare a lipid film (step 103); hydrating the lipid film to form multi-lamellar vesicles (step 104); and downsizing the multi-lamellar vesicles to obtain a nano-sized liposome composition (step 105). The prepared liposomes may have a neutral, negative or positive charge at physiological conditions. The prepared liposomes may have a selected mean particle diameter in the size range of about 40 nm to about 250 nm

Referring to step 101, the prepared complex may be a phytosome and it may be a lipophilic complex. The lipophilic complex may be prepared by reacting the at least one polyphenolic compound, the first phospholipid, and the organic solvent or a mixture of solvents, such as dioxane, acetone, n-hexane. According to some implementations, once the complex is prepared, it may be isolated by precipitation with non-solvents (e.g., aliphatic hydrocarbons), lyophilization or by spray-drying.

It should be noted that the terms “phytosome” and “liposome” have structurally different meanings. A “phytosome” is a unit of several molecules bonded together, while a “liposome” is an aggregate of many phospholipid molecules that may enclose active phyto-molecules or “phytosome”, but without specifically bonding to them.

Since, liposomes may generally encapsulate hydrophilic molecules in their aqueous core and incorporate lipophilic molecules in their lipid bilayers, thus, forming the complex may help increasing the absorption of polyphenolic compound, which are typically not readily soluble in aqueous or lipophilic phase. Forming the complex containing at least one type of polyphenolic compound, in addition to saving high content of the at least one polyphenol in lipid bilayers of liposomes, may provide a sustained release nano-carrier upon arrival in target organs, for example, in the tumor area. Moreover, the stability of liposomes may be improved by coating their surfaces with a protecting hydrophilic polymer, for example polyethylene glycol. This strategy may result in enhancement of liposome circulation time and prevention of their rapid clearance.

Moving on to step 102, the admixture, in addition to contain the aforementioned complex, may include cholesterol, one or more polyethylene glycol (PEG)-phospholipid conjugated molecule or other hydrophilic polymers-phospholipid conjugated molecules, and one or more type of phospholipid molecules as second phospholipid. According to an implementation, the first and the second phospholipid may be selected from the group consisting of phosphatidylcholine (PC), phosphatidylethanolamine (PE), or phosphatidylserine (PS). In related implementations, the first and the second phospholipid may be selected from a group consisting of egg phosphatidylcholine (EPC), soy phosphatidylcholine (SPC), hydrogenated soy phosphatidylcholine (HSPC), Dipalmitoylphosphatidylcholine (DPPC), and dimyristoylphosphatidylcholine (DMPC) or all other type of phospholipids suitable for use in aliposome composition.

The prepared liposomes of the present disclosure, may exhibit suitable circulation time and accumulate within tumors via EPR (Enhanced Permeability and Retention) effect, which may result on their applying for various purposes, including but not limited to: inhibition of tumor growth, improvement of vascular function, and modulation of the immune system. In some other aspect, they also may be used in the treatment of inflammatory and autoimmune conditions.

In some implementations, the prepared liposome may cause greater desired effects after parental administration than oral administration, the exemplary prepared liposome may be administrated intravenously, although its oral administrating may be possible as well.

According to one example implementation, various polyphenolic compound may be incorporated in the prepared liposome including: anthocyanidins, catechins, flavonoids (flavanones, flavones, flavonols, isoflavones, etc.), hydroxybenzoic acids, hydroxycinnamic acids, lignans, stilbenes, tannins (proanthocyanidines), monophenols, capsaicinoids, curcumin and derivatives thereof.

Moving on to step 103, preparing the lipid film may take place via dissolving the admixture obtained in step 102 into one organic compound, for example chloroform. For preparing the lipid film according to one example implementation, the organic solvents may be further removed. In an exemplary implementation, removing the organic solvents, according to step 103 may be carried out via rotary evaporation. In some example implementations, freeze-drying may further be employed for complete removing of organic solvents.

Moving on to step 104, multi-lamellar vesicles (MLVs) may be prepared by hydrating and dispersing the prepared lipid film with a hydrating liquid. In some example implementations, the solvent used for preparing the MLVs may be HEPES.

Moving on to step 105, example operations for downsizing the liposomes may include sonicating, extruding, filtering, or a combination of these techniques.

The following examples represent methods and techniques for carrying out aspects of the present disclosure. It should be understood that numerous modifications may be made without departing from the intended scope of the disclosure.

### Example 1: Forming Sylimarin or Sylibin Complex

In an example implementation, silymarin as the active polyphenolic compounds of milk thistle plant may be encapsulated in the prepared liposome of the present disclosure. In general, silymarin composed of silybin (also known as silybinin) and small amounts of its stereoisomers, such as isosilybin A, isosilybin B, silychristin, isosilychristin, and silydianin. Above all, silymarin and its main component silybin, are well known for their hepatoprotective, antioxidant and chemo-protective effects. It has been found that they also have significant anti-neoplastic effects in a variety of in vitro and in vivo cancer models, including skin, breast, lung, colon, bladder and prostate.

In spite of aforesaid advantages, silymarin compounds are multi-ring hydrophobic molecules with low lipophilicity and indeed they are too large to be incorporated in a large extent into lipid bilayer membranes without disturbing their integrity. Moreover, despite certain methods known for people skilled in the art, which may result in silymarin incorporating into various liposome compositions, leakage out of silymarin molecules during storage is observed. Besides, silymarin characteristically, due to its short half-life, may have rapid metabolism after its absorption into blood stream. Therefore, the silymarin incorporation into the example complex, as an example initially step of preparing the liposome composition, according to one and more aspect of the present disclosure results in its increased bioavailability, cellular uptake, increased drug-release pattern time, high-efficacy, as well as avoiding drug administration frequency.

### Preparation of Silymarin—Soy-Phosphatidylcholine (SPC) Complexes

For forming the exemplary complex, a suspension of 482 mg silymarin (about 1 millimoles) in 10 milliliters acetone, was treated with 1540-2310 milligram (about 2-3 millimoles) of soybean phosphatidycholine (SPC) (Lipoid S 100®) overnight under stirring condition. Then the reaction mixture was concentrated in vacuum to a volume of about 2 milliliters. After being diluted with a solvent, for example n-hexane in amount of 10 milliliters, the pale yellow complex is precipitated and then it is collected by filtration after one night and it is dried under vacuum at 40° C.

### Preparation of Silybin—Soy-Phosphatidylcholine (SPC) Complexes

A suspension of 482 mg silybin (about 1 millimoles) in 10 milliliters acetone was treated with 1540-2310 milligram (about 2-3 millimoles) of soybean phosphatidycholine (SPC) (Lipoid S 100®) as an example first phospholipid under stirring condition. Then the reaction mixture was concentrated in vacuum to a volume of about 2 milliliters. After being diluted with a solvent, for example n-hexane in amount of 10 milliliters, the pale yellow complex is precipitated and then it is collected by filtration after one night and it is dried under vacuum at 40° C.

### Example 2: Preparation of Liposomes

Liposomes according to present example implementation were prepared by admixing at second phospholipid, such as Dipalmitoylphosphatidylcholine (DPPC), cholesterol, polyethylene glycol (PEG)-phospholipid conjugated molecules, such as 1,2-distearoyl-sn-glycero-3-phosphoethanolamine-N-[amino (polyethylene glycol)-2000] (mPEG2000-DSPE), alpha tocopherol as an antioxidant, and one of the complex prepared in the prior step (silybin—SPC complexes or silymarin—SPC complexes). The example molar ratios of components for preparation of liposome composition according to present disclosure including DPPC:cholesterol:PEG-phospholipid:antioxidant:and the complex containing silybin or silymarin, may be in range of 20:5:1:0.02:2 to 25:6:2:0.08:3 and more preferably may be 21:5.6:1.4:0.05:3.

Liposomes are prepared using lipid film hydration and extrusion methods. Briefly, the lipids and the complex containing polyphenolic compound were dissolved in chloroform. Then in order to formation a thin layer lipid film; the organic solvent was removed by rotary evaporation. For complete removal of the solvents; the lipid film was freeze-dried. Then, the lipid film was hydrated and dispersed in HEPES 10 mM (millimolar) containing about 10% sucrose (pH 7), using a vortex at 50° C. The resulting multi-lamellar vesicles (MLVs), were then downsized by a 3-5 minutes sonication and then extrusion through stacked 200 and 80 nm polycarbonate filters with a mini-extruder apparatus. The particle size and zeta potential of the liposomes were determined by a particle size analyzer.

### Example 3: Physicochemical Characterization of Liposomes

In this example, the physicochemical characterization was carried out for the liposomes-incorporated polyphenolic compounds, the preparing of which are described in more detail in connection with Example 1 and Example 2.

The mean particle diameters, polydispersity index (which is denoted as PDI herein after), zeta potential and encapsulation percentage of silymarin liposome and silybin liposome encapsulated are presented and set forth in TABLE 1. Each value represents Mean±standard deviation (n=3). As seen from TABLE 1, there is no significant difference in Z-average of prepared liposomal formulations using silybin and silymarin. Further reference to TABLE 1, emphasizes high encapsulation of the prepared liposome formulations especially in silymarin liposome in which about 84% of silymarin was encapsulated into the liposome composition.

TABLE 1
Physical properties of silybin liposomes and silymarin liposomes.

| | | Size by | | Zeta | |
|---|---|---|---|---|---|
| | Z-Average | number | | potential | |
| Formulation | (nm) | (nm)^{a} | PDI | (mV) | Encapsulation % |
| Silybin | 141.46 ± 2.75 | 77.97 ± 3.51 | 0.231 ± 0.01 | −16.5 | 45.98 ± 2.55 |
| liposomes | | | | | |
| Silymarin | 129.1 ± 1.4 | 83.5 ± 2.48 | 0.165 ± 0.01 | −13.7 | 84.52 ± 5.63 |
| liposomes | | | | | |

^{a}Mean ± SD (n = 3).

### Example 4: Cell Viability Assay

In this example, cell viability was determined by using methyl thiazolyl tetrazolium bromide (MTT) assay. For this purpose, a stock solution of MTT dye in Phosphate Buffer Saline (PBS) was prepared, phosphate buffer saline (5 mg/ml in PBS). Then the solution was filtered through a 0.45 μm filter. The prepared solution was stored at −20° C. for frequent use.

Cell plates prepared at 1×10³ 4 T1 cells/well by adding 200 μl of a 5×10³ cells/ml suspension to each well of a 96-well cell culture plate. Each plate included negative control wells having medium and no cell counts and 4T1 cell cultured wells containing 200 μl RPMI cell culture medium used as positive control in each plate. After overnight incubation of plates at 37° C., the medium carefully was aspirated off, without any removal of 4T1 cells. Then it was replaced with fresh medium (200 μl) containing up to 100 μl of each formulation. The plates incubated at 37° C., 5% CO2 for 24, 48 and 72 hours. Four hours before the end of incubation, the medium carefully was aspirated off and replaced by 100 μl FCS free cell cultured medium containing 10 μl of MTT solution. In living cells, mitochondrial dehydrogenases may convert soluble MTT yellow dye to an insoluble purple formazan precipitate by cleavage of the tetrazolium ring. This conversion has been used to develop an assay system for measurement of cell viability. The produced insoluble formazan was dissolved by adding 200 μl DMSO and its optical density (OD) was read with a multi-well scanning spectrophotometer at a wavelength of 570 nano meters.

The percentage of cytotoxicity was calculated according to following formulas:

% Cytotoxicity=100×[1−(mean absorbance of drug treated cells−mean absorbance of negative control cells)/(mean absorbance of positive control cells−mean absorbance of negative control cells)]

% viability=100−% Cytotoxity

### Example 5: In Vitro Studies

In this example, time-dependent cytotoxicities of liposome-encapsulated silymarin, and silybin liposome were measured on 4T1 cancer cells. Half maximal inhibitory concentration (denoted as IC50s) of the prepared liposomes during the various incubation periods, which were 24, 48 and 72 hours, are presented and set forth in TABLE 2

TABLE 2
Tumor cell specific cytotoxicity of liposome-encapsulated silymarin, and
liposome-encapsulated silybin in different incubation period
against 4T1 cancer cells.

| | IC50 (μg/ml ± SD) | | |
|---|---|---|---|
| Formulation | 24 Hours | 48 Hours | 72 Hours |
| Silybin Liposomes | 104.52 ± 26.4 | 36.22 ± 8.6 | 24.5 ± 6.5 |
| Silymarin Liposomes | 138.51 ± 75.9 | 46.63 ± 6.3 | 28.36 ± 7.9 |

According to TABLE 2, a significant decrease in LC50 is observed during 48 hours' incubation in either silybin or silymarin liposomes than 24-hours incubation, whilst there is no significant difference between 48 hours and 72 hours' incubation period. This emphasizes higher cytotoxity in long incubation period for both silymarin and silybin liposomes. Moreover, silybin represented higher cytotoxity than silymarin liposomes since silybin is the most active derivative of silymarin compounds.

### Example 6: In-Vivo Studies

FOR IN-VIVO studies according to one example implementation, female BALB/c mice (aged 8 weeks, 18-20 g) were acquired. The mice were kept in an animal house at 21° C. in a colony room 12/12 hours light/dark cycle with free access to water and animal food. All mice received humane care in compliance with institutional guidelines. On day 0, BALB/c mice were given subcutaneous injections of 4T1 cells (2.5×10⁵ cells per mouse) or TUBO cells (5×10⁵ cells per mouse) in the right hind flank. Then tumors were allowed to grow until mice had palpable tumors (11 days), and animals were divided into 3 different treatments with 5-6 mice per each group. Liposomal formulations were injected at a 10 mg/kg drug (Silybin, Silymarin or Doxil) dose i.v. via the lateral tail vein. Starting on the day of the treatment, the animals' weight, tumor volume and overall health were monitored on 3 occasions a week for 60 days. Three dimensions of tumor were measured with calipers and tumor volume was calculated via the following formula:

tumor volume=(height×length×width)×0.52 cm³

For ethical considerations, mice were sacrificed due to decrease in body weight (>15% loss) or tumor enlargement (more than 2 cm in one dimension) or declining health. The time to reach end point (TTE) for each mouse was calculated from the equation of the line obtained by exponential regression of the tumor growth curve. Subsequently, the percent of tumor growth delay (% TGD) was calculated based on the difference between the mean TTE of treatment group (T) and the mean TTE of the control group (C), (% TGD=[(T−C)/C]×100).

### In Vivo Study Results

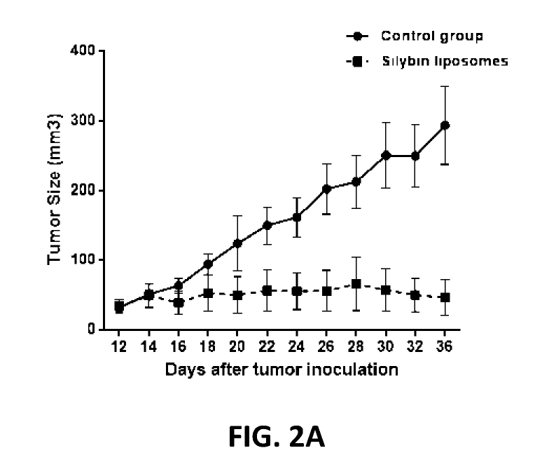

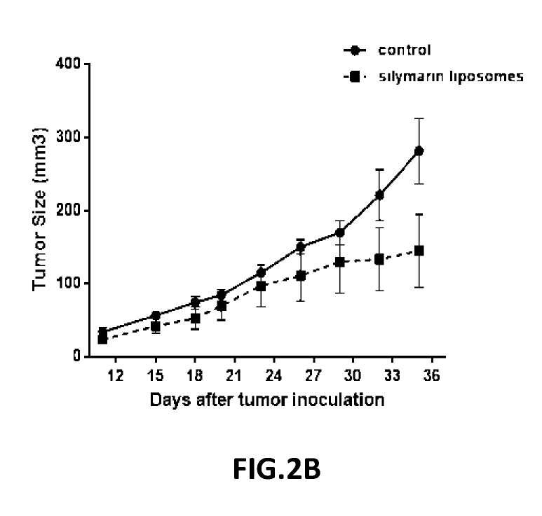

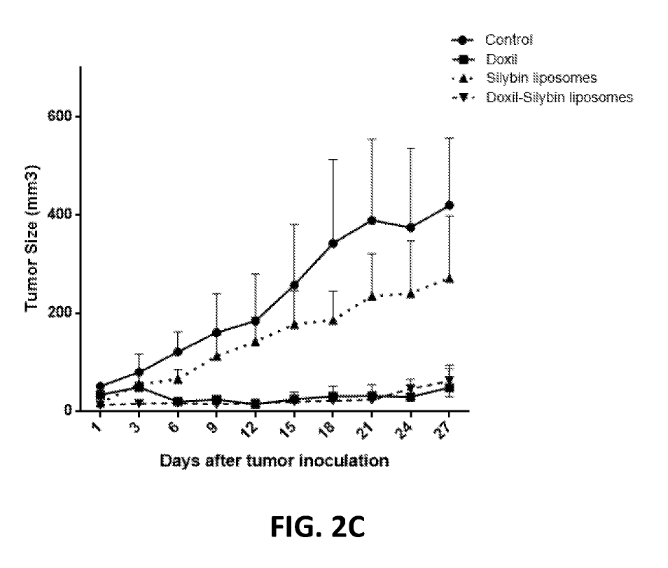

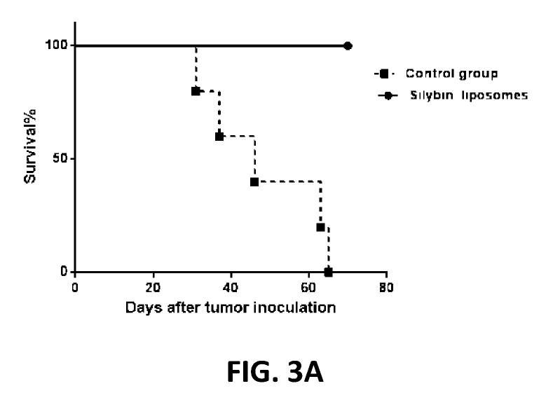

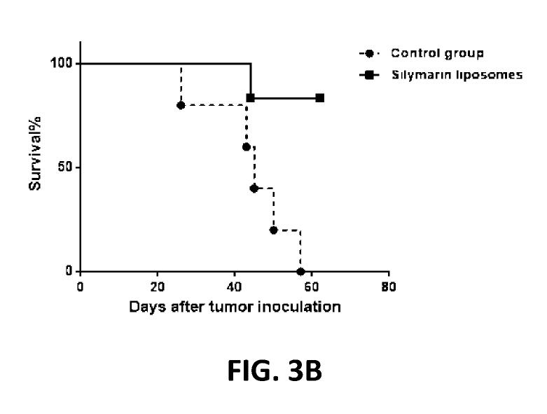

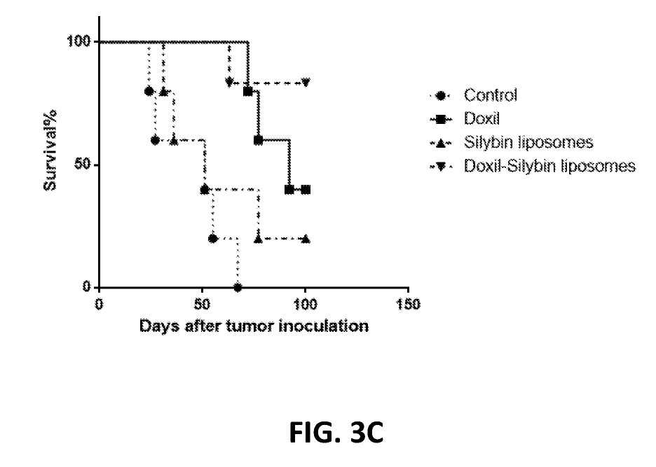

To determine the cytotoxic efficacy of silymarin liposomes and silybin liposomes, their anti-tumor activities were assessed in a mouse 4T1 breast tumor model. After injection of prepared liposomes according to examples 1 and 2, tumor size and survival rate were monitored 3 times a week for 60 days. Because of tumor necrosis in 4T1 breast tumor model, data until day 35 was utilized for evaluation of formulations efficacy on tumor growth, as shown in FIGS. 2 and 3

FIG. 2A illustrates results of tumor size monitoring experiments in female BALB/c mice bearing 4T1 breast tumor after, for example parenteral administration of a single dose of 10 mg/kg silybin, while FIG. 2B illustrates the same result after administrating the same amount of silymarin or dextrose 5% on day 11 after tumor inoculation. FIG. 2C illustrates the results after administrating a single dose of 10 mg/kg Doxil or silybin liposomes (alone or in combination with each other) at the same dose simultaneously and also dextrose 5% on day 11 after tumor inoculation in female BALB/c mice bearing TUBO breast tumor.

Referring now to FIG. 2A and FIG. 2B, these figures illustrate that anti-tumor efficacy silymarin and silybin liposome formulations in treated mice were significantly more than control animals on day 35 (p<0.001 for silybin liposome and p<0.01 for silymarin liposome). Referring to FIG. 2C, the results show that although anti-tumor efficacy of Doxil and Doxil-Silybin liposomes was significantly more than control animals (p<0.001), efficacy of silybin liposome formulations in treated mice (when used alone not in combination with Doxil) were not significantly more than control animals on day 27.

The results of in vivo survival experiments in female BALB/c mice bearing 4T1 and TUBO breast tumor after intravenous administration of a single dose of 10 mg/kg in prepared liposome compositions on day 11 after tumor inoculation are illustrated in FIG. 3A, FIG. 3B, and FIG. 3C. With this regard, after intravenous administration of the exemplary liposomal formulations on day 11 post tumor inoculation, mice were monitored and euthanized for determining of decreased body weight (more than 15%) or tumor enlargement (tumor volume more than 1000 mm³ or 2 cm in one dimension).

FIG. 3A illustrates results of in vivo survival experiments in female BALB/c mice bearing 4T1 breast tumor after intravenous. Administration of a single dose of 10 mg/kg silybin on day 11 after tumor inoculation, while FIG. 3B and FIG. 3C illustrates the same, but FIG. 3B represents the results after intravenous administration of same amount of silymarin liposome. FIG. 3C represents the results after intravenous administration a single dose of 10 mg/kg of Doxil or silybin liposomes alone or in combination with each other at the same dose simultaneously. For comparing the result with control group, dextrose 5% was given to the control group in same experimental conditions. According to FIGS. 3A and 3B, even 80 days after incubation, 100 percent survival was observed when silybin liposomes were used, whilst in case of silymarin liposome, after 50 days, the survival percentage reached to almost 80% and remained on this percent. The survival percent of the control group reached to zero, on around 60 days after tumor incubation. Referring to FIG. 3C, even 100 days after inoculation, 83% percent survival was observed when Doxil-silybin liposomes were used simultaneously while in case of Doxil alone, after 100 days, the survival percentage for this group reached to almost 40% and for silybin liposomes alone reached to 20%. Therefore, there is a significant difference between survival percent in control group with Doxil alone (p<0.001) and Doxil-silybin liposomes combination (p<0.001). The survival percent of the control group reached to zero, on around 40 days after tumor inoculation.

TABLEs 3, 4, and 5 represent and set forth some criteria concerning the therapeutic efficacy of liposomal formulations in 4T1 and TUBO mouse model. The data on this table include median survival time, time to reach endpoint (TTE) and the percentage of tumor growth delay (% TGD). Results showed that silybin and Silymarin nano liposomal formulations may increase the median survival time and also they may induce tumor growth delay of 32.26% in silybin liposome and 29.71% in silymarin nano-liposomes treated groups.

The results of TABLE 5 in TUBO tumor bearing mice model showed that liposomal formulations may induce tumor growth delay of 31.7% in silybin liposome and 96.87% in Doxil and 109.44% in Doxil-Silybin liposomes combination treated groups. These results indicated that although silybin liposomes cannot reduce the Median survival significantly in TUBO tumor bearing mice, combination therapy using silybin liposomes and Doxil, may induce improvement in survival status of treated mice.

TABLE 3
Therapeutic efficacy data of liposomal formulations of silybin in mice
earing 4T1 tumor.

| Group | Median survival(day)^{a} | TTE^{b} | TGD %^{c} |
|---|---|---|---|
| Control (Dextrose 5% | 46 | 48.4 ± 15.22 | — |
| Silybin liposomes | Undefinedd | 70 | 44.63 |
| | | (tumor free) | |

^{a}Median survival time.
^{b}Time to reach end point.
^{c}Tumor growth delay.
^{d}100% were alive at the day 70

TABLE 4
Therapeutic efficacy data of liposomal formulations of silymarin in mice
bearing 4T1 tumor

| Group | Median survival (day)^{a} | TTE^{b} | TGD %^{c} |
|---|---|---|---|
| Control (Dextrose | 45 | 44.2 ± 11.51 | — |
| 5%) | | | |
| Silymarin liposomes | Undefinedd | 57.33 ± 6.53 | 29.71 |

^{a}Median survival time.
^{b}Time to reach end point.
^{c}Tumor growth delay.
^{d} more than 80% were alive at the day 60

TABLE 5
Therapeutic efficacy data of Doxil and silybin liposomal formulations
alone and in combination with each otherr in mice bearing TUBO tumor

| | Median survival | | |
|---|---|---|---|
| Group | (day)^{a} | TTE^{b} | TGD %^{c} |
| Control (Dextrose 5%) | 51 | 44.8 ± 18.6 | — |
| Silybin liposomes | 51 | 59 ± 29.07 | 31.7 |
| Doxil (doxorubicin | 92 | 88.2 ± 13.04 | 96.87 |
| liposomes) | | | |
| Doxil-silybin liposomes | Undefined | 93.8 ± 15.1 | 109.44 |

^{a}Median survival time.
^{b}Time to reach end point.
^{c}Tumor growth delay.
^{d}>80% of mice were alive at the day 100

It will be understood that the terms and expressions used herein have the ordinary meaning as is accorded to such terms and expressions with respect to their corresponding respective areas of inquiry and study, except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” and any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

While the present application has been illustrated by the description of the examples thereof, and while the example has been described in detail, it is not the intention of the applicant to restrict or in any way limit the scope of the appended claims to such detail. Additional advantages and modifications will readily appear to those skilled in the art. Therefore, the application in its broader aspects is not limited to the specific details, representative apparatus and method, and illustrative examples shown and described. Accordingly, departures may be made from such details without departure from the breadth or scope of the applicant's concept. Furthermore, although the present application has been described in connection with a number of exemplary embodiments and implementations, the present application is not so limited but rather covers various modifications and equivalent arrangements, which fall within the purview of the appended claims.

What is claimed is:

1. A method for treating tumors, the method comprising:
preparing a liposome composition, the preparing comprising:
forming a complex comprising at least one polyphenolic compound and a first phospholipid;
admixing the complex with at least one second phospholipid, cholesterol, an antioxidant and a PEG-Phospholipid, wherein the first phospholipid and the second phospholipid are selected from the group consisting of phosphatidylcholine (PC), phosphatidylethanolamine (PE), phosphatidylserine (PS), and combinations thereof;
using the admixture to prepare multi-lamellar vesicles; and
downsizing the prepared multi-lamellar vesicles to reach a nano-sized liposome composition, the downsizing comprising a three to five minutes sonication and subsequent extrusion through stacked 200 and 80 nm polycarbonate filters; and
intravenously administering the nano-sized liposome composition.

2. The method according to claim 1, wherein using the admixture to prepare multi-lamellar vesicles comprises:
dissolving the admixture into a solvent and removing organic solvents to prepare a lipid film; and
hydrating the lipid film.

3. The method according to claim 1, wherein forming a complex comprising at least one polyphenolic compound and a first phospholipid comprises reacting the at least one polyphenolic compound with the first phospholipid.

4. The method according to claim 1, wherein a molar ratio of the first phospholipid to the at least one polyphenolic compound in the complex is in the range of 0.5:1 to 3:1.

5. The method according to claim 1, wherein the first phospholipid is soybean phosphatidycholine (SPC).

6. The method according to claim 1, wherein the solvent is selected from the group consisting of acetone, dioxane, and n-hexane.

7. The method according to claim 1, wherein the PEG-phospholipid is 1, 2-distearoyl-sn-glycero-3-phosphoethanolamine-N-[amino (polyethylene glycol)-X] (mPEGX-DSPE), wherein the X is in a range of 500 to 5000.

8. The method according to claim 1, wherein the polyphenolic compound is selected from the group consisting of anthocyanidins, catechins, flavonoids, hydroxybenzoic acids, hydroxycinnamic acids, lignans, stilbenes, tannins, monophenols, capsaicinoids, curcumin and derivations thereof.

9. The method according to claim 1, wherein a molar ratio of the second phospholipid:cholesterol:PEG-phospholipid:antioxidant:and the complex containing polyphenolic compound is in the range of between 20:5:1:0.02:2 and 25:6:2:0.08:3.

10. A liposome composition for delivering at least one polyphenolic compound to a target via intravenous administration, the liposome composition comprising:
a complex containing at least one polyphenolic compound and a first phospholipid, a second phospholipid, cholesterol, a PEG-phospholipid, and an antioxidant, wherein:
the first phospholipid and the second phospholipid are selected from the group consisting of phosphatidylcholine (PC), phosphatidylethanolamine (PE), phosphatidylserine (PS), and combinations thereof
a molar ratio of the first phospholipid to the at least one polyphenolic compound is in the range of 0.5:1 to 3:1;
a molar ratio of the at least one polyphenolic compound:second phospholipid:cholesterol:PEG-phospholipid:and antioxidant is in range of 2:20:5:1:0.02 to 3:25:6:2:0.08.

11. The composition according to claim 10, wherein the first phospholipid is soybean phosphatidycholine (SPC).

12. The composition according to claim 10, wherein the second phospholipid is selected from the group consisting of egg phosphatidylcholine (EPC), SPC, hydrogenated soy phosphatidylcholine (HSPC), Dipalmitoylphosphatidylcholine (DPPC), and dimyristoylphosphatidylcholine (DMPC).

13. The composition according to claim 10, wherein the at least one polyphenolic compound is selected from the group consisting of anthocyanidins, catechins, flavonoids (flavanones, flavones, flavonols, isoflavones, etc.), hydroxybenzoic acids, hydroxycinnamic acids, lignans, stilbenes, tannins (proanthocyanidines), monophenols, capsaicinoids, curcumin and derivatives thereof.
